(19) United States
(12) Patent Application Publication
(10) Pub. No.: US 2019/0192397 A1
(43) Pub. Date: Jun. 27, 2019

(54) HAIR OILS FOR CONDITIONING KERATIN FIBRES

(71) Applicant: Henkel AG & Co. KGaA, Duesseldorf (DE)

(72) Inventors: Edith Von Aspern, Hanstedt (DE); Jens Delowsky, Norderstedt (DE); Thomas Schroeder, Hamburg (DE)

(73) Assignee: Henkel AG & Co. KGaA, Duesseldorf (DE)

(21) Appl. No.: 16/228,139

(22) Filed: Dec. 20, 2018

(30) Foreign Application Priority Data
Dec. 21, 2017 (DE) 10 2017 223 528.4

(51) Int. Cl. A61K 8/31; A61K 8/9789; A61K 8/37; A61Q 5/12

(52) U.S. Cl. CPC A61K 8/31; A61K 8/9789; A61K 2800/5922; A61Q 5/12; A61K 2800/34; A61K 8/37

(57) ABSTRACT

The present disclosure relates to cosmetic agents which are based on natural oils, are preferably free from silicones and preferably are present in the form of a hair oil, and use thereof for the care of keratin fibres.

### CROSS-REFERENCE TO RELATED APPLICATION

This application claims priority to German Patent Application No. 10 2017 223 528.4, filed Dec. 21, 2017, which is incorporated herein by reference in its entirety.

### TECHNICAL FIELD

The present disclosure relates to cosmetic agents which are based on natural oils, are preferably free from silicones and preferably are present in the form of a hair oil, and use thereof for the care of keratin fibres.

### BACKGROUND

Hair conditioners having a caring effect primarily include quats (quaternary ammonium compounds), silicone oils, fatty acids and polymer(s). For a few years now, the field of hair cosmetics has been developing hair oils that primarily include silicone oils. In addition, the hair oils are used as “leave-in products”, that is to say the formulations remain in the hair without subsequent rinsing. The consumer thus saves time and water. The hair oils enjoy an advantageous sustainability from this point of view. The success of hair oils of this kind on the market has been very high.

The search is ongoing for new combinations that can ensure a high level of care, a perceptible care effect, and good stability. Within the scope of sustainability, increasing value is currently being placed on raw materials of plant origin or having good biodegradability. In this regard, silicone oils are a constant source of criticism. The good level of care, however, should not be compromised. The greatest difficulty in replacing silicone oils in hair oils lies in finding natural oils or natural raw materials that do not weigh down the hair or make it greasy.

Besides natural ambient influences, human hair is also exposed to a range of further, in particular cosmetic stresses. These stresses, which burden the hair, for example include hair dyeing and hair shaping, for example as a result of permanent waving. Cosmetic haircare agents are used in order to reduce the disadvantageous effects of the (ambient) influences that compromise the hair structure, but also in order to maintain and improve the natural hair structure. An essential active substance in many of these cosmetic agents is constituted by the organosilicon compounds, in particular the silicones such as trisiloxanes, which are exemplified by caring properties. The disadvantages of these silicones are the reduced penetration of active substances and auxiliaries into the hair caused by wetting of the hair surface, and the increased difficulty in styling the hair, likewise caused by the wetting of the hair surface. The provision of care agents that have a low silicone content or are free from silicone is therefore a relevant objective in the field of hair cosmetics.

The user of ester oils in hair-conditioning agents is described in international patent application WO 2010/063565 A1 (Henkel).

Document EP 2 623 088 A2 (Henkel) discloses haircare agents that contain at least one ester oil and at least one isoparaffin from the group of isodecane, isoundecane, isododecane, isotridecane and isotetradecane. Mixtures of ester oils and polymer quaternary ammonium compounds are additionally described, as are also combinations of both cosmetic agents.

### BRIEF SUMMARY

Cosmetic agents are provided herein. In an embodiment, a cosmetic agent includes, in a cosmetically acceptable carrier,
a) one or more alkanes chosen from the group of undecane, dodecane, tridecane, or mixtures thereof;
b) at least one ester oil chosen from the group of dicarboxylic acid esters, carbonate esters, diol esters, or mixtures thereof, and
crambe abyssinica seed oil.

In another embodiment, a cosmetic agent includes, in a cosmetically acceptable carrier,
a) one or more alkanes chosen from the group of undecane, dodecane, tridecane, or mixtures thereof,
b) at least one ester oil chosen from the group of dicarboxylic acid esters, carbonate esters, diol esters, or mixtures thereof,
c) crambe abyssinica seed oil,
d) at least one ester oil chosen from the group of tri-fatty acid esters of saturated and/or unsaturated linear and/or branched fatty acids with glycerol, and
e) at least one ester oil chosen from the group of esters of C₆ to C₃₀ fatty acids with C2 to C₃₀ fatty alcohols. The agent is free from silicones.

In another embodiment, a cosmetic agent includes, in a cosmetically acceptable carrier,
a) a mixture of undecane, dodecane, and tridecane,
b) at least one ester oil chosen from the group of dicarboxylic acid esters, carbonate esters, diol esters, or mixtures thereof, wherein at least one ester oil b) comprises an ester oil selected from the group of esters of carbonic acid with C₆₋₁₂ alkanols,
c) crambe abyssinica seed oil,
d) at least one ester oil chosen from a mixture of tri-fatty acid esters of caprylic acid and capric acid with glycerol, and
e) at least one ester oil chosen from the group of esters of C₆ to C₃₀ fatty acids with C2 to C₃₀ fatty alcohols. The agent is free from silicones.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure is to provide cosmetic agents that make the co-use of organosilicon compounds, in particular silicones, superfluous and yet nevertheless maintain the performance range of the silicone-containing formulations. The weighing-down of the hair and the greasy condition of the treated hair often occurring with the use of silicones should be avoided. In particular, the object of the present disclosure is that it is enabling the use of renewable raw materials or the incorporation of natural oil in a cosmetic agent without the agent weighing down the hair or making it greasy.

The object is achieved as contemplated herein by a cosmetic agent containing, in a cosmetically acceptable carrier,

a) one or more alkanes from the group of undecane, dodecane, tridecane,
b) at least one ester oil from the group of dicarboxylic acid esters, carbonate esters and diol esters,
c) crambe abyssinica seed oil.

It has been found that by combining one or more alkanes with a) at least ester oil and b) crambe abyssinica seed oil, cosmetic agents are produced that do not weigh down the hair treated therewith or make said hair greasy and in addition ensure good combability and a good level of hair shine. Cosmetic agents, in particular haircare agents, are thus produced which demonstrate an excellent level of care and in particular provide good shine properties, an improved appearance and preferably improved regeneration of the keratin fibres.

In particular, cosmetic agents that surprisingly provide improved care in respect of their detangling ability and combability, feel, shine and anti-frizz properties, without weighing down the hair, are produced. Especially by the use of crambe abyssinica seed oil, the cosmetic agent as contemplated herein gains even more advantages in respect of its care properties. In addition, the use has a strong sustainable aspect and enables the incorporation of plant oils based on renewable raw materials. In particular, the use of plant-based alkanes is possible, whereby the use of petrochemical constituents can be reduced or avoided.

As component a) the cosmetic agents contain one or more alkanes from the group of undecane, dodecane, tridecane. Individual alkanes or mixtures of two or more of the mentioned alkanes can be used.

For example, mixtures of undecane and dodecane, mixtures of dodecane and tridecane, mixtures of undecane and tridecane, or mixtures of undecane, dodecane and tridecane can be used.

Cosmetic agents as contemplated herein that contain a mixture of undecane and tridecane or exclusively dodecane as alkane demonstrate particularly advantageous effects in combination with the oil bodies b) and c).

The proportion by weight of the undecane, dodecane and tridecane in the total weight of all alkanes used in the cosmetic agent is preferably more than about 62% by weight, preferably more than about 72% by weight, particularly preferably more than about 82% by weight, and in particular more than about 92% by weight. It is particularly preferred if the proportion by weight of the dodecane and tridecane in the total weight of all alkanes used in the cosmetic agent is more than about 60% by weight, preferably more than about 70% by weight, particularly preferably more than about 80% by weight, and in particular more than about 90% by weight.

An alkane mixture that is particularly preferred as contemplated herein is commercially available under the name Cetiol® Ultima. A further preferred alkane mixture is commercially available under the name Parafol 12-97.

The at least one ester oil b) is selected from the group of dicarboxylic acid esters, carbonate esters and diol esters.

These are understood to be:
dicarboxylic acid esters such as di-n-butyl adipate, di-(2-ethylhexyl) adipate, di-(2-ethylhexyl) succinate and di-isotridecylacelaate and
diol esters such as ethylene glycol dioleate, ethylene glycol-di-isotridecanoate, propylene glycol-di(2-ethylhexanoate), propylene glycol-di-isostearate, propylene glycol-di-pelargonate, butanediol-di-isostearate, neopentyl glycol dicaprylate, and
symmetrical, asymmetrical or cyclic esters of carbonic acid with fatty alcohols, such as glycerol carbonate or dicaprylyl carbonate (Cetiol® CC).

The at least one ester oil b), however, is preferably selected from symmetrical, asymmetrical or cyclic esters of carbonic acid with fatty alcohols. The at least one ester oil b) is particularly preferably selected from esters of carbonic acid with C₆₋₁₂ alkanols, in particular C₇₋₉ alkanols. The esters are particularly preferably symmetrical carbonic acid esters with alkanols, especially dicaprylyl carbonate (carbonic acid esters with n-octyl alcohol).

Mixtures of two or more of the above-mentioned ester oils of component b) can also be used as component b). Here, the presence of at least one symmetrical, asymmetrical or cyclic esters of carboxylic acid with fatty alcohols is preferred.

In mixtures of ester oils b), a majority of symmetrical, asymmetrical or cyclic esters of carbonic acid is particularly preferably present with alcohols. In accordance with one embodiment of the present disclosure the at least one ester oil b) contains merely symmetrical, asymmetrical or cyclic esters of carbonic acid with fatty alcohols. The at least one ester oil b) is very particularly preferably dicaprylyl carbonate (Cetiol® CC).

The cosmetic agent as contemplated herein additionally contains seed oil from the plant crambe abyssinica, which is also referred to as Abyssinian Kale. The oil is known as a cosmetic additive under the INCI name Crambe Abyssinica Seed Oil. Crambe abyssinica seed oil usually contains erucic acid, oleic acid, linoleic acid and linolenic acid as main constituents.

The cosmetic agents as contemplated herein can contain the components a), b) and c) in any suitable amounts.

The cosmetic agent as contemplated herein preferably contains from about 10 to about 60% by weight of component a), particularly preferably from about 15 to about 50% by weight, very particularly preferably from about 20 to about 40% by weight, and in particular from about 25 to about 35% by weight of component a).

The cosmetic agent as contemplated herein preferably contains from about 10 to about 60% by weight of component b), particularly preferably from about 15 to about 50% by weight, very particularly preferably from about 20 to about 40% by weight, and in particular from about 25 to about 35% by weight of component b).

The cosmetic agent as contemplated herein preferably contains from about 1 to about 40% by weight of component c), particularly preferably from about 3 to about 35% by weight, very particularly preferably from about 5 to about 30% by weight, and in particular from about 10 to about 25% by weight of component c).

In a preferred embodiment the cosmetic agent as contemplated herein additionally contains at least one ester oil d) from the group of tri-fatty acid esters of saturated and/or unsaturated linear and/or branched fatty acids with glycerol. The at least one ester oil d) is preferably selected from tri-fatty acid esters of saturated linear and/or branched C₆₋₁₂ carboxylic acids with glycerol. Here, the use of linear C₆₋₁₂ carboxylic acids, in particular C₈₋₁₀ carboxylic acids, is particularly preferred. The at least one ester oil d) is particularly preferably a mixture of tri-fatty acid esters of caprylic acid and capric acid with glycerol, preferably in a ratio by weight of caprylic acid triglyceride to capric acid triglyceride of from about 3:1 to about 1:1.

In a further preferred embodiment the cosmetic agent as contemplated herein additionally contains at least one ester oil e) from the group of esters of C₆-C₃₀ fatty acids with C₂-C₃₀ fatty alcohols. Component e) can be contained instead of component d) or as an additional component. The monoesters of fatty acids with alcohols with from about 2 to about 24 C atoms are preferred. Examples of used fatty acid components in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachinic acid, gadoleic acid, behenic acid and erucic acid and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof.

Particularly preferred as contemplated herein are isopropyl myristate (Rilanit® IPM), isononanoic acid C16-18 alkyl esters (Cetiol® SN), 2-ethylhexyl palmitate (Cegesoft® 24), stearic acid 2-ethylhexyl esters (Cetiol® 868), cetyloleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl esters (Cetiol® A), di-n-butyl adipate (Cetiol® B), myristyl myristate (Cetiol® MM), cetearyl isononanoate (Cetiol® SN), oleic acid decyl esters (Cetiol® V).

Mixtures of two or more of the above-mentioned ester oils of component e) can also be used as component e). The at least one ester oil e) is very particularly preferably isopropyl myristate.

The cosmetic agent as contemplated herein can also contain fatty acid partial glycerides, that is to say monoglycerides, diglycerides and technical mixtures thereof, instead of component e) or as additional constituent. With use of technical products, small amounts of triglycerides may still be contained due to the production process. The partial glycerides preferably follow formula (D4-1I),

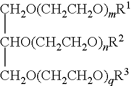

in which R1, R2 and R3 independently of one another stand for hydrogen or for a linear or branched, saturated and/or unsaturated acyl group with from about 6 to about 22, preferably from about 12 to about 18 carbon atoms, with the provision that at least one of these groups stands for an acyl group and at least of these groups stands for hydrogen. The sum (m+n+q) stands for 0 or numbers from about 1 to about 100, preferably for 0 or from about 5 to about 25. R1 preferably stands for an acyl group, and R2 and R3 preferably stand for hydrogen, and the sum (m+n+q) is 0. Typical examples are mono- and/or diglycerides based on caproic, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachinic acid, gadoleic acid, behenic acid and erucic acid and technical mixtures thereof. Oleic acid monoglycerides are preferably used.

The cosmetic agents as contemplated herein can contain the components a), b), c), d) and e) in any suitable amounts. The components a), b) and c) are contained as contemplated herein. The components d) and e) can each be contained as optional constituents. Cosmetic agents as contemplated herein for example contain mixtures of a), b) and c), mixtures of a), b), c) and d), mixtures of a), b), c) and e), or mixtures of a), b), c), d) and e) without limitation thereto.

The cosmetic agent as contemplated herein preferably contains from about 10 to about 60% by weight of component a), particularly preferably from about 15 to about 50% by weight, very particularly preferably from about 20 to about 40% by weight, and in particular from about 25 to about 35% by weight of component a).

The cosmetic agent as contemplated herein preferably contains from about 10 to about 60% by weight of component b), particularly preferably from about 15 to about 50% by weight, very particularly preferably from about 20 to about 40% by weight, and in particular from about 25 to about 35% by weight of component b).

The cosmetic agent as contemplated herein preferably contains from about 1 to about 40% by weight of component c), particularly preferably from about 3 to about 35% by weight, very particularly preferably from about 5 to about 30% by weight, and in particular from about 10 to about 25% by weight of component c).

The cosmetic agent as contemplated herein preferably contains from about 1 to about 35% by weight of component d), particularly preferably from about 3 to about 30% by weight, very particularly preferably from about 5 to about 25% by weight, and in particular from about 7 to about 20% by weight of component d).

The cosmetic agent as contemplated herein preferably contains from about 1 to about 25% by weight of component e), particularly preferably from about 2 to about 20% by weight, very particularly preferably from about 3 to about 15% by weight, and in particular from about 4 to about 10% by weight of component e).

In a preferred embodiment the cosmetic agent additionally contains from about 0.1 to about 0.8% by weight, preferably from about 0.2 to about 0.6% by weight perfume.

Based on the cosmetic agent, the total amount of which makes about 100% by weight, the sum of components a), b), c), d) and e) preferably accounts for from about 10 to about 99.9% by weight, preferably from about 50 to about 99% by weight, in particular from about 90 to about 99.8% by weight.

Preferred cosmetic agents, in relation to their total weight, contain the components a), b), c), d) and e) in proportions by weight above about 90% by weight, preferably above about 94% by weight, and in particular above about 98% by weight. In a preferred embodiment the cosmetic agents consist essentially of components a), b), c), d) and e).

The cosmetic agent as contemplated herein can be adapted to the particular hair quality by the selection of suitable amounts of components b), c), d) and e). Formulations for the treatment of fine, normal or thick hair can thus be obtained selectively.

By use of the cosmetic agents as contemplated herein it is possible to dispense with organosilicon compounds, in particular silicones, such as trisiloxanes, in the cosmetic agents. Preferred embodiments as contemplated herein are therefore exemplified in that
the cosmetic agent, in relation to its total weight, contains less than about 5.0% by weight, preferably less than about 2.0% by weight, preferably less than about 0.5% by weight, and in particular no trisiloxane; or
the cosmetic agent, in relation to its total weight, contains less than about 5.0% by weight, preferably less than about 2.0% by weight, preferably less than about 0.5% by weight, and in particular no dimethicone; or
the cosmetic agent, in relation to its total weight, contains less than about 5.0% by weight, preferably less than about 2.0% by weight, preferably less than about 0.5% by weight, and in particular no amodimethicone; or
the cosmetic agent, in relation to its total weight, contains less than about 5.0% by weight, preferably less than about 2.0% by weight, preferably less than about 0.5% by weight, and in particular no cyclomethicone; or
the cosmetic agent, in relation to its total weight, contains less than about 5.0% by weight, preferably less than about 2.0% by weight, preferably less than about 0.5% by weight, and in particular no organosilicon compounds.

The cosmetic agent is preferably free from organosilicon compounds, in particular free from silicones, trisiloxanes, and silicone oils.

In a preferred embodiment the cosmetic agent as contemplated herein has the following composition (values in % by weight relate to the total weight of the cosmetic agent):

alkanes: from about 10 to about 60%
caprylic acid/capric acid triglyceride: from about 1 to about 35%
dicaprylyl carbonate: from about 10 to about 60%
isopropyl myristate:_{—} from about 1 to about 25%
crambe abyssinica seed oil: from about 1 to about 40%
perfume from about 0.1 to about 0.8%

In a further preferred embodiment the cosmetic agent as contemplated herein has the following composition (values in % by weight relate to the total weight of the cosmetic agent):

alkanes: from about 15 to about 50%
caprylic acid/capric acid triglyceride: from about 3 to about 30%
dicaprylyl carbonate: from about 15 to about 50%
isopropyl myristate:_{—} from about 2 to about 20%
crambe abyssinica seed oil: from about 3 to about 35%
perfume from about 0.2 to about 0.6%

In a further preferred embodiment the cosmetic agent as contemplated herein has the following composition (values in % by weight relate to the total weight of the cosmetic agent):

alkanes: from about 20 to about 40%
caprylic acid/capric acid triglyceride: from about 5 to about 25%
dicaprylyl carbonate: from about 20 to about 40%
isopropyl myristate:_{—} from about 3 to about 15%
crambe abyssinica seed oil: from about 5 to about 30%
perfume from about 0.2 to about 0.6%

In a further preferred embodiment the cosmetic agent as contemplated herein has the following composition (values in % by weight relate to the total weight of the cosmetic agent):

alkanes: from about 25 to about 35%
caprylic acid/capric acid triglyceride: from about 7 to about 20%
dicaprylyl carbonate: from about 25 to about 35%
isopropyl myristate:_{—} from about 4 to about 10%
crambe abyssinica seed oil: from about 10 to about 25%
perfume from about 0.2 to about 0.6%

The composition of some preferred cosmetic agents can be inferred from the following tables (values in % by weight relate to the total weight of the cosmetic agent unless specified otherwise). In the following compositions the ester oil b) is in particular dicaprylyl carbonate. The ester oil d) is in particular caprylic acid/capric acid triglyceride. In particular the ester oil b) is dicaprylyl carbonate and the ester oil d) is caprylic acid/capric acid triglyceride. Each of the following compositions can additionally contain from about 1 to about 25% by weight ester oil e), in particular isopropyl myristate. In particular, in each of the following compositions the ester oil b) can be dicaprylyl carbonate, the ester oil d) can be caprylic acid/capric acid triglyceride, and the composition can contain from about 1 to about 25% by weight ester oil e), in particular isopropyl myristate.

| Formula | 1 | 2 | 3 | 4 | 5 | 6 |
|---|---|---|---|---|---|---|
| alkanes a) | 10 to 60 | 10 to 60 | 10 to 60 | 10 to 60 | 10 to 60 | 10 to 60 |
| ester oil b) | 10 to 60 | 10 to 60 | 10 to 60 | 10 to 60 | 15 to 50 | 15 to 50 |
| crambe oil c) | 1 to 40 | 3 to 35 | 5 to 30 | 10 to 25 | 1 to 40 | 3 to 35 |
| misc | to 100 | to 100 | to 100 | to 100 | to 100 | to 100 |
| Formula | 7 | 8 | 9 | 10 | 11 | 12 |
| alkanes a) | 10 to 60 | 10 to 60 | 10 to 60 | 10 to 60 | 10 to 60 | 10 to 60 |
| ester oil b) | 15 to 50 | 15 to 50 | 20 to 40 | 20 to 40 | 20 to 40 | 20 to 40 |
| crambe oil c) | 5 to 30 | 10 to 25 | 1 to 40 | 3 to 35 | 5 to 30 | 10 to 25 |
| misc | to 100 | to 100 | to 100 | to 100 | to 100 | to 100 |
| Formula | 13 | 14 | 15 | 16 | 17 | 18 |
| alkanes a) | 10 to 60 | 10 to 60 | 10 to 60 | 10 to 60 | 20 to 40 | 20 to 40 |
| ester oil b) | 25 to 35 | 25 to 35 | 25 to 35 | 25 to 35 | 10 to 60 | 10 to 60 |
| crambe oil c) | 1 to 40 | 3 to 35 | 5 to 30 | 10 to 25 | 1 to 40 | 3 to 35 |
| misc | to 100 | to 100 | to 100 | to 100 | to 100 | to 100 |
| Formula | 19 | 20 | 21 | 22 | 23 | 24 |
| alkanes a) | 20 to 40 | 20 to 40 | 20 to 40 | 20 to 40 | 20 to 40 | 20 to 40 |
| ester oil b) | 10 to 60 | 10 to 60 | 15 to 50 | 15 to 50 | 15 to 50 | 15 to 50 |
| crambe oil c) | 5 to 30 | 10 to 25 | 1 to 40 | 3 to 35 | 5 to 30 | 10 to 25 |
| misc | to 100 | to 100 | to 100 | to 100 | to 100 | to 100 |
| Formula | 25 | 26 | 27 | 28 | 29 | 30 |
| alkanes a) | 20 to 40 | 20 to 40 | 20 to 40 | 20 to 40 | 20 to 40 | 20 to 40 |
| ester oil b) | 20 to 40 | 20 to 40 | 20 to 40 | 20 to 40 | 25 to 35 | 25 to 35 |
| crambe oil c) | 1 to 40 | 3 to 35 | 5 to 30 | 10 to 25 | 1 to 40 | 3 to 35 |
| misc | to 100 | to 100 | to 100 | to 100 | to 100 | to 100 |

| Formula | 31 | 32 |
|---|---|---|
| alkanes a) | 20 to 40 | 20 to 40 |
| ester oil b) | 25 to 35 | 25 to 35 |
| crambe oil c) | 5 to 30 | 10 to 25 |
| misc | to 100 | to 100 |

| Formula | 33 | 34 | 35 | 36 | 37 | 38 |
|---|---|---|---|---|---|---|
| alkanes a) | 10 to 60 | 10 to 60 | 10 to 60 | 10 to 60 | 10 to 60 | 10 to 60 |
| ester oil b) | 10 to 60 | 10 to 60 | 10 to 60 | 10 to 60 | 15 to 50 | 15 to 50 |
| crambe oil c) | 1 to 40 | 3 to 35 | 5 to 30 | 10 to 25 | 1 to 40 | 3 to 35 |
| ester oil d) | 1 to 35 | 1 to 35 | 1 to 35 | 1 to 35 | 1 to 35 | 1 to 35 |
| misc | to 100 | to 100 | to 100 | to 100 | to 100 | to 100 |
| | 39 | 40 | 41 | 42 | 43 | 44 |
| alkanes a) | 10 to 60 | 10 to 60 | 10 to 60 | 10 to 60 | 10 to 60 | 10 to 60 |
| ester oil b) | 15 to 50 | 15 to 50 | 20 to 40 | 20 to 40 | 20 to 40 | 20 to 40 |
| crambe oil c) | 5 to 30 | 10 to 25 | 1 to 40 | 3 to 35 | 5 to 30 | 10 to 25 |
| ester oil d) | 1 to 35 | 1 to 35 | 1 to 35 | 1 to 35 | 1 to 35 | 1 to 35 |
| misc | to 100 | to 100 | to 100 | to 100 | to 100 | to 100 |
| Formula | 45 | 46 | 47 | 48 | 49 | 50 |
| alkanes a) | 10 to 60 | 10 to 60 | 10 to 60 | 10 to 60 | 20 to 40 | 20 to 40 |
| ester oil b) | 25 to 35 | 25 to 35 | 25 to 35 | 25 to 35 | 10 to 60 | 10 to 60 |
| crambe oil c) | 1 to 40 | 3 to 35 | 5 to 30 | 10 to 25 | 1 to 40 | 3 to 35 |
| ester oil d) | 1 to 35 | 1 to 35 | 1 to 35 | 1 to 35 | 1 to 35 | 1 to 35 |
| misc | to 100 | to 100 | to 100 | to 100 | to 100 | to 100 |
| Formula | 51 | 52 | 53 | 54 | 55 | 56 |
| alkanes a) | 20 to 40 | 20 to 40 | 20 to 40 | 20 to 40 | 20 to 40 | 20 to 40 |
| ester oil b) | 10 to 60 | 10 to 60 | 15 to 50 | 15 to 50 | 15 to 50 | 15 to 50 |
| crambe oil c) | 5 to 30 | 10 to 25 | 1 to 40 | 3 to 35 | 5 to 30 | 10 to 25 |
| ester oil d) | 1 to 35 | 1 to 35 | 1 to 35 | 1 to 35 | 1 to 35 | 1 to 35 |
| misc | to 100 | to 100 | to 100 | to 100 | to 100 | to 100 |
| Formula | 57 | 58 | 59 | 60 | 61 | 62 |
| alkanes a) | 20 to 40 | 20 to 40 | 20 to 40 | 20 to 40 | 20 to 40 | 20 to 40 |
| ester oil b) | 20 to 40 | 20 to 40 | 20 to 40 | 20 to 40 | 25 to 35 | 25 to 35 |
| crambe oil c) | 1 to 40 | 3 to 35 | 5 to 30 | 10 to 25 | 1 to 40 | 3 to 35 |
| ester oil d) | 1 to 35 | 1 to 35 | 1 to 35 | 1 to 35 | 1 to 35 | 1 to 35 |
| misc | to 100 | to 100 | to 100 | to 100 | to 100 | to 100 |

| Formula | 63 | 64 |
|---|---|---|
| alkanes a) | 20 to 40 | 20 to 40 |
| ester oil b) | 25 to 35 | 25 to 35 |
| crambe oil c) | 5 to 30 | 10 to 25 |
| ester oil d) | 1 to 35 | 1 to 35 |
| misc | to 100 | to 100 |

| Formula | 65 | 66 | 67 | 68 | 69 | 70 |
|---|---|---|---|---|---|---|
| alkanes a) | 10 to 60 | 10 to 60 | 10 to 60 | 10 to 60 | 10 to 60 | 10 to 60 |
| ester oil b) | 10 to 60 | 10 to 60 | 10 to 60 | 10 to 60 | 15 to 50 | 15 to 50 |
| crambe oil c) | 1 to 40 | 3 to 35 | 5 to 30 | 10 to 25 | 1 to 40 | 3 to 35 |
| ester oil d) | 5 to 25 | 5 to 25 | 5 to 25 | 5 to 25 | 5 to 25 | 5 to 25 |
| misc | to 100 | to 100 | to 100 | to 100 | to 100 | to 100 |
| | 71 | 72 | 73 | 74 | 75 | 76 |
| alkanes a) | 10 to 60 | 10 to 60 | 10 to 60 | 10 to 60 | 10 to 60 | 10 to 60 |
| ester oil b) | 15 to 50 | 15 to 50 | 20 to 40 | 20 to 40 | 20 to 40 | 20 to 40 |
| crambe oil c) | 5 to 30 | 10 to 25 | 1 to 40 | 3 to 35 | 5 to 30 | 10 to 25 |
| ester oil d) | 5 to 25 | 5 to 25 | 5 to 25 | 5 to 25 | 5 to 25 | 5 to 25 |
| misc | to 100 | to 100 | to 100 | to 100 | to 100 | to 100 |
| Formula | 77 | 78 | 79 | 80 | 81 | 82 |
| alkanes a) | 10 to 60 | 10 to 60 | 10 to 60 | 10 to 60 | 20 to 40 | 20 to 40 |
| ester oil b) | 25 to 35 | 25 to 35 | 25 to 35 | 25 to 35 | 10 to 60 | 10 to 60 |
| crambe oil c) | 1 to 40 | 3 to 35 | 5 to 30 | 10 to 25 | 1 to 40 | 3 to 35 |
| ester oil d) | 5 to 25 | 5 to 25 | 5 to 25 | 5 to 25 | 5 to 25 | 5 to 25 |
| misc | to 100 | to 100 | to 100 | to 100 | to 100 | to 100 |
| Formula | 83 | 84 | 85 | 86 | 87 | 88 |
| alkanes a) | 20 to 40 | 20 to 40 | 20 to 40 | 20 to 40 | 20 to 40 | 20 to 40 |
| ester oil b) | 10 to 60 | 10 to 60 | 15 to 50 | 15 to 50 | 15 to 50 | 15 to 50 |
| crambe oil c) | 5 to 30 | 10 to 25 | 1 to 40 | 3 to 35 | 5 to 30 | 10 to 25 |
| ester oil d) | 5 to 25 | 5 to 25 | 5 to 25 | 5 to 25 | 5 to 25 | 5 to 25 |
| misc | to 100 | to 100 | to 100 | to 100 | to 100 | to 100 |
| Formula | 89 | 90 | 91 | 92 | 93 | 94 |
| alkanes a) | 20 to 40 | 20 to 40 | 20 to 40 | 20 to 40 | 20 to 40 | 20 to 40 |
| ester oil b) | 20 to 40 | 20 to 40 | 20 to 40 | 20 to 40 | 25 to 35 | 25 to 35 |
| crambe oil c) | 1 to 40 | 3 to 35 | 5 to 30 | 10 to 25 | 1 to 40 | 3 to 35 |
| ester oil d) | 5 to 25 | 5 to 25 | 5 to 25 | 5 to 25 | 5 to 25 | 5 to 25 |
| misc | to 100 | to 100 | to 100 | to 100 | to 100 | to 100 |

| Formula | 95 | 96 |
|---|---|---|
| alkanes a) | 20 to 40 | 20 to 40 |
| ester oil b) | 25 to 35 | 25 to 35 |
| crambe oil c) | 5 to 30 | 10 to 25 |
| ester oil d) | 5 to 25 | 5 to 25 |
| misc | to 100 | to 100 |

The cosmetic agent as contemplated herein can be used in any suitable fields of application. The agents are preferably hair treatment agents, in particular hair oils. The cosmetic agent is preferably present in the form of a hair oil.

The hair oil is particularly preferably applied in the form of a sprayed oil and is sprayed onto the hair. The spray can be a pump spray, or the spraying can be achieved with the aid of propellants. Suitable propellants (propellant gases) are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluoroethane, heptafluoro-n-propane, perfluoroethane, monochlorodifluoromethane, 1,1-difluoroethane, more specifically either individually or in combination. Hydrophilic propellant gases such as carbon dioxide, can also be used advantageously in the context of the present disclosure, if the proportion of hydrophilic gases is selected to be low and a lipophilic propellant gas (for example propane/butane) is present in excess. Propane, n-butane, isobutane, and mixtures of said propellant gases are particularly preferred. It has emerged that the use of n-butane as the sole propellant gas can be particularly preferred as contemplated herein.

Vessels made of metal (aluminium, tinplate, tin), protected or non-splintering plastic, or glass that is externally coated with plastic may be used as compressed gas containers; compressive strength and breaking strength, corrosion resistance, ease of filling, as well as aesthetic aspects, handling, printability, etc., play a role in their selection.

The present disclosure thus also relates to the use of the cosmetic agent for the care of keratin fibres.

As discussed, the agents as contemplated herein are suitable in particular for haircare. A further subject of the present application is therefore a method for the treatment of keratin fibres, exemplified in that a cosmetic agent as contemplated herein is applied to the dried and/or damp keratin fibres.

The hair treatment agent as contemplated herein is preferably used directly before, during or after an oxidative or surfactant hair treatment. In the context of the present disclosure, the expression “directly before the oxidative or surfactant hair treatment” is understood to mean an application that directly follows the oxidative or surfactant hair treatment, wherein the hair treatment agent as contemplated herein has been rinsed beforehand from the hair or preferably has been left on the hair, and the hair is preferably still wet.

The expression “after the oxidative or surfactant hair treatment” in the context of the present disclosure is understood to mean an application which either directly follows the oxidative or surfactant hair treatment, wherein the hair treatment agent as contemplated herein is applied to the preferably still wet, towel-dried hair once the agent with oxidative or surfactant effect has been rinsed out, or is applied to the dry or wet hair only after several hours or days. In both cases the hair treatment agent as contemplated herein can be rinsed out again after a reaction time of a few seconds up to about 45 minutes, or can remain fully on the hair.

The effect of the hair treatment agent as contemplated herein develops even during the oxidative or surfactant hair treatment and surprisingly continues even after the hair treatment agent as contemplated herein has been washed out vigorously.

A further subject of the present application is the use of a cosmetic agent as contemplated herein for the care of keratin fibres. What is claimed here in particular is the use of a cosmetic agent as contemplated herein
to improve the wet and dry combability of keratin fibres,
to improve the shine of keratin fibres,
to improve the moisture balance of keratin fibres,
to protect the keratin fibres against oxidative damage, in particular during the course of oxidative hair treatment, so as to prevent the keratin fibres from becoming greasy,
to increase the washing fastness of dyed keratin fibres,
to attain increased hairstyle durability over at least about 48 h, in particular beyond about 24 h,
to improve what is known as the curl retention of keratin fibres,
to improve the feel of keratin fibres,
to improve the regeneration of keratin fibres.

Combability is understood as contemplated herein to mean both the combability of the wet fibres and the combability of the dry fibres. The combing work involved or the force applied during the process of combing a collective of fibres is used as a measure for combability. The measurement parameters can be assessed in a sensory manner by a person skilled in the art or can be quantified using measuring devices.

An oxidative hair treatment is defined as contemplated herein to mean the effect on hair of an oxidative cosmetic agent containing at least one oxidising agent in a cosmetic carrier.

Besides the active substances described above, the cosmetic agents as contemplated herein can contain further ingredients. The group of these further ingredients includes, in particular, the cosmetically effective active substances, auxiliaries and additives.

A first group of optional ingredients is formed by the substances that form oil bodies. Examples of these are plant oils, liquid paraffin oils and synthetic hydrocarbons, and di-n-alkyl ethers with a total of between from about 12 and about 36 C atoms and fatty alcohols:
plant oils. Examples of such oils are amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice bran oil, sea buckthorn pulp oil, sea buckthorn seed oil, sesame oil, Shea butter, soy oil, sunflower oil, grapeseed oil, walnut oil, wheat germ oil, wild rose oil and the liquid components of coconut oil. The proportion by weight of the plant oil in the total weight of preferred cosmetic agents is from about 0.1 to about 10% by weight, preferably from about 0.2 to about 8.0% by weight, and in particular from about 0.4 to about 5.0% by weight;
liquid paraffin oils and synthetic hydrocarbons and di-n-alkyl ethers with a total between from about 12 and about 36 C atoms, in particular from about 12 to about 24 C atoms, such as di-noctyl ether, di-n-decyl ether, di-n-nonyl ether, di-n-undecyl ether, di-n-dodecyl ether, n-hexyl-noctyl ether, n-octyl-n-decyl ether, n-decyl-n-undecyl ether, n-undecyl-n-dodecyl ether and n-hexyl-n-undecyl ether and di-tert-butyl ether, di-iso-pentyl ether, di-3-ethyldecyl ether, tert.-butyl-n-octyl ether, iso-pentyl-n-octyl ether and 2-methyl-pentyl-n-octyl ether. The compounds 1,3-di-(2-ethyl-hexyl)-cyclohexane (Cetiol® S) and di-n-octyl ether (Cetiol® OE) obtainable as commercial products can be preferred;
fatty alcohols. The group of preferred fatty alcohols includes C₈ to C₂₂ fatty alcohols, preferably C₁₀ to C₂₀ fatty alcohols, and in particular C₁₂ to C₁₈ fatty alcohols. The proportion by weight of the fatty alcohol in the total weight of preferred cosmetic agents is from about 0.1 to about 10% by weight, preferably from about 0.2 to about 8.0% by weight, and in particular from about 0.4 to about 5.0% by weight.

The total amount of the ingredients of the cosmetic agents is 100% by weight.

Besides the active substances described above, the cosmetic agents as contemplated herein can contain further ingredients. The group of these further ingredients includes, in particular, the cosmetically effective active substances, auxiliaries and additives.

Suitable active substances, auxiliaries and additives are in particular additional care substances. The agent for example can contain at least one protein hydrolysate and/or one of the derivatives thereof as care substance. Protein hydrolysates are product mixtures that are obtained by acid-catalysed, base-catalysed or enzymatically catalysed breakdown of proteins. The term “protein hydrolysates” is understood as contemplated herein to also mean total hydrolysates and also individual amino acids and derivatives thereof as well as mixtures of different amino acids. The molecular weight of the protein hydrolysates usable as contemplated herein lies between about 75, the molecular weight for glycine, and about 200,000 daltons, and the molecular weight is preferably from about 75 to about 50,000 daltons, and very particularly preferably from about 75 to about 20,000 daltons.

As care substance, the agent as contemplated herein can also contain at least one vitamin, a provitamin, a vitamin precursor and/or one of the derivatives thereof. Here, vitamins, provitamins and vitamin precursors that are usually assigned to the groups A, B, C, E, F and H are preferred as contemplated herein.

Similarly to the addition of glycerol and/or propylene glycol, the addition of panthenol increases the flexibility of the polymer film formed with application of the agent as contemplated herein.

As care substance, the agents as contemplated herein can also contain at least one plant extract, but also monosaccharides or oligosaccharides and/or lipids.

Particularly preferred cosmetic agents as contemplated herein are preferably formulated as application mixtures with a low water content. The water content of preferred cosmetic agents, in relation to the total weight of said agents, is less than about 10% by weight, preferably less than about 5.0% by weight, particularly preferably less than about 1.0% by weight, and in particular less than about 0.1% by weight, wherein very particularly preferred cosmetic agents contain no water.

### Example

All specified amounts relate to parts by weight. The following formulation was provided with use of known production methods.

Hair Oil1

### Undecane/tridecane: 30.0

caprylic acid/capric acid triglyceride: 15.5
dicaprylyl carbonate: 29.0
isopropyl myristate
perfume: 0.5

For example, Cetiol® Ultimate can be used as undecane/tridecane mixture. A product available under the UNCI name Caprylic/Capric Triglyceride can be used as caprylic acid/capric acid triglyceride.

For example, Cetiol® CC, which is di-n-octyl carbonate, can be used as dicapryl carbonate.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent comprising, in a cosmetically acceptable carrier,
a) one or more alkanes chosen from the group of undecane, dodecane, tridecane, or mixtures thereof;
b) at least one ester oil chosen from the group of dicarboxylic acid esters, carbonate esters, diol esters, or mixtures thereof, and
c) crambe abyssinica seed oil.

2. The cosmetic agent according to claim 1, wherein component a) comprises two or more alkanes chosen from the group of undecane, dodecane, or tridecane.

3. The cosmetic agent according to claim 1, further comprising at least one ester oil d) from the group of tri-fatty acid esters of saturated and/or unsaturated linear and/or branched fatty acids with glycerol.

4. The cosmetic agent according to claim 3, wherein the at least one ester oil d) comprises a mixture of tri-fatty acid esters of caprylic acid and capric acid with glycerol.

5. The cosmetic agent according to claim 1, further comprising at least one ester oil e) chosen from the group of esters of C₆ to C₃₀ fatty acids with C2 to C₃₀ fatty alcohol.

6. The cosmetic agent according to claim 1, wherein the at least one ester oil b) is selected from the group of symmetrical, asymmetrical or cyclic esters of carbonic acid with fatty alcohols.

7. The cosmetic agent according to claim 1, wherein in the cosmetic agent, in relation to the total amount of components a), b) and c), which gives about 100% by weight, comprises
from about 10 to about 60% by weight of component a),
from about 10 to about 60% by weight of component b), and
from about 1 to about 40% by weight of component c) are present.

8. The cosmetic agent according to claim 1, said agent being free from silicones.

9. The cosmetic agent according to claim 1, in the form of a hair oil.

10. (canceled)

11. The cosmetic agent according to claim 1, wherein component a) comprises a mixture of undecane, dodecane and tridecane.

12. The cosmetic agent according to claim 1, further comprising at least one ester oil d) chosen from the group of tri-fatty acid esters of saturated linear and/or branched C₆₋₁₂ carboxylic acids with glycerol.

13. The cosmetic agent according to claim 5, wherein the at least one ester oil (e) comprises isopropyl myristate.

14. The cosmetic agent according to claim 1, wherein the at least one ester oil b) comprises an ester oil selected from the group of esters of carbonic acid with C₆₋₁₂ alkanols.

15. The cosmetic agent according to claim 1, wherein the at least one ester oil b) comprises dicaprylyl carbonate.

16. A cosmetic agent comprising, in a cosmetically acceptable carrier,
a) one or more alkanes chosen from the group of undecane, dodecane, tridecane, or mixtures thereof,
b) at least one ester oil chosen from the group of dicarboxylic acid esters, carbonate esters, diol esters, or mixtures thereof,
c) crambe abyssinica seed oil,
d) at least one ester oil chosen from the group of tri-fatty acid esters of saturated and/or unsaturated linear and/or branched fatty acids with glycerol, and
e) at least one ester oil chosen from the group of esters of C₆ to C₃₀ fatty acids with C₂ to C₃₀ fatty alcohols,
wherein said agent is free from silicones.

17. The cosmetic agent according to claim 16, wherein in the cosmetic agent, in relation to the total amount of components a), b) and c), which gives about 100% by weight, comprises
from about 10 to about 60% by weight of component a),
from about 10 to about 60% by weight of component b), and
from about 1 to about 40% by weight of component c) are present.

18. A cosmetic agent comprising, in a cosmetically acceptable carrier,
a) a mixture of undecane, dodecane, and tridecane,
b) at least one ester oil chosen from the group of dicarboxylic acid esters, carbonate esters, diol esters, or mixtures thereof, wherein at least one ester oil b) comprises an ester oil selected from the group of esters of carbonic acid with C₆₋₁₂ alkanols,
c) crambe abyssinica seed oil,
d) at least one ester oil chosen from a mixture of tri-fatty acid esters of caprylic acid and capric acid with glycerol, and
e) at least one ester oil chosen from the group of esters of C₆ to C30 fatty acids with C2 to C₃₀ fatty alcohols,
wherein said agent is free from silicones.

19. The cosmetic agent according to claim 18, wherein the at least one ester oil (e) comprises isopropyl myristate.

20. The cosmetic agent according to claim 19, wherein the at least one ester oil b) comprises dicaprylyl carbonate.

21. The cosmetic agent according to claim 18, wherein in the cosmetic agent, in relation to the total amount of components a), b) and c), which gives about 100% by weight, comprises
from about 10 to about 60% by weight of component a),
from about 10 to about 60% by weight of component b), and
from about 1 to about 40% by weight of component c) are present.
